(19) United States
(12) Patent Application Publication
(10) Pub. No.: US 2022/0317520 A1
(43) Pub. Date: Oct. 6, 2022

(54) DISPLAY SYSTEM

(71) Applicant: Japan Display Inc., Tokyo (JP)

(72) Inventor: Masashi TAKAHATA, Tokyo (JP)

(21) Appl. No.: 17/687,877

(22) Filed: Mar. 7, 2022

(30) Foreign Application Priority Data
Mar. 30, 2021 (JP) 2021-058188

(51) Int. Cl. G02F 1/13357; G02F 1/1333; G02F 1/1335; A61L 2/00

(52) U.S. Cl. CPC G02F 1/133615; G02F 1/133342; G02F 1/133308; G02F 1/133614; A61L 2/0047; A61L 2202/11; G09G 3/3413

(57) ABSTRACT

A display device according to an embodiment of the present invention includes a display panel for displaying images, a lighting device provided corresponding to a first side face of the display panel, and a housing surrounding the display panel and the lighting device, the housing including a first opening provided in a front face corresponding to the display panel and a second opening in a side face. A light emitted from the lighting device enters the display panel from the first side face, a first portion of the incident light is emitted from the first opening, and a second portion of the incident light is emitted from the second opening through at least one portion of a second side face in the display panel corresponding to the first side face.

This application is based upon and claims the benefit of priority from the prior Japanese Patent Application 2021-058188, filed on Mar. 30, 2021, the entire contents of which are incorporated herein by reference.

### FIELD

An embodiment of the present invention relates to a display system.

### BACKGROUND

A liquid crystal display system includes a liquid crystal display panel and a backlight arranged on the back of the liquid crystal display panel. For example, Japanese Laid Open Patent No. 2014-102295 discloses a technique of synchronizing with the rewriting of an image displayed by a liquid crystal display panel, forming a backlight arranged on the back of the liquid crystal display panel by a polymer-dispersed liquid crystal panel, and blinking light by synchronizing with a period in which light is scattered by the polymer-dispersed liquid crystal panel.

### SUMMARY

A display system according to an embodiment of the present invention includes a display panel for displaying images, a lighting device provided corresponding to a first side face of the display panel, and a housing surrounding the display panel and the lighting device, the housing including a first opening provided in a front face corresponding to the display panel and a second opening in a side face. A light emitted from the lighting device enters the display panel from the first side face, a first portion of the incident light is emitted from the first opening, and a second portion of the incident light is emitted from the second opening through at least one portion of a second side face in the display panel corresponding to the first side face.

### BRIEF DESCRIPTION OF THE DRAWINGS

FIG. 1 is a perspective view showing a display system according to an embodiment of the present invention;

FIG. 2 is a schematic diagram showing the inside of a housing of a display system according to an embodiment of the present invention;

FIG. 3 is a block diagram of a display system according to an embodiment of the present invention;

FIG. 4 is a timing chart for explaining a timing at which a light emitting element according to an embodiment of the present invention emits light.

FIG. 5 is a cross-sectional view when a lighting device and a display panel according to an embodiment of the present invention are viewed from a side face;

FIG. 6 is an enlarged cross-sectional view of a part of a display panel according to an embodiment of the present invention;

FIG. 7 is an enlarged cross-sectional view of a part of a display panel according to an embodiment of the present invention;

FIG. 8 is a schematic diagram showing the inside of a housing of a display system according to an embodiment of the present invention;

FIG. 9 is a block diagram of a display system according to an embodiment of the present invention;

FIG. 10 is a specific example of a display system according to an embodiment of the present invention;

FIG. 11 is a specific example of a display system according to an embodiment of the present invention;

FIG. 12 is a schematic diagram showing the inside of a housing of a display system according to an embodiment of the present invention;

FIG. 13 is a block diagram of a display system according to an embodiment of the present invention;

FIG. 14 is a specific example of a display system according to an embodiment of the present invention;

FIG. 15A is a specific example of a display system according to an embodiment of the present invention; and

FIG. 15B is a specific example of a display system according to an embodiment of the present invention.

### DESCRIPTION OF EMBODIMENTS

Embodiments of the present invention will be described below with reference to the drawings. The disclosure is merely an example, and those skilled in the art could easily conceive of appropriate changes while maintaining the gist of the invention are such changes are naturally included in the scope of the invention. For the sake of clarity of explanation, although the drawings may be schematically represented with respect to widths, thicknesses, shapes, and the like of the respective portions in comparison with actual embodiments, they are merely an example and do not limit the interpretation of the present invention. In this specification and each of the drawings, the same symbols (or symbols denoted by A, B, and the like) are given to the same elements as those described previously with reference to the preceding drawings, and a detailed description thereof may be omitted as appropriate. In addition, the letters “first” and “second” attached to each element are convenient labels used to distinguish each element and have no further meaning unless otherwise stated.

Furthermore, in the detailed description of the present invention, in defining the positional relationship between one component and another, the terms “above” and “below” include not only the case of being positioned directly above or below one component, but also the case of interposing another component therebetween, unless otherwise specified.

A normal liquid crystal display panel is surrounded by a housing. Since light emitted from a light source (lighting device) is emitted to the outside only from a portion corresponding to the display panel, there is a problem that the light emitted from the light source is not effectively used.

The present disclosure provides a display system with high light utilization efficiency.

### First Embodiment

### (1-1. Configuration of Display System)

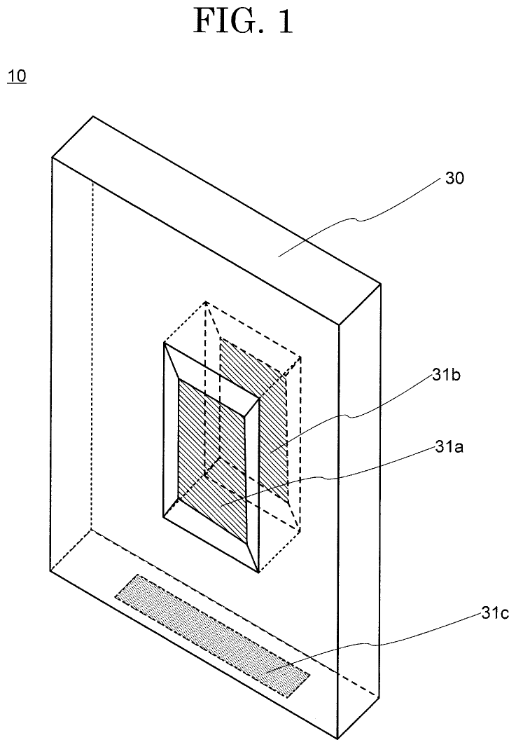

Hereinafter, a display system according to an embodiment of the present invention will be described. FIG. 1 is a perspective view of a display system 10. In FIG. 1, the display system 10 includes a housing 30, an opening 31a (also referred to as a first opening), an opening 31b (also referred to as a third opening), and an opening 31c (also referred to as a second opening). The opening 31a is provided in a front face of the housing 30. The opening 31b is provided in a back face of the housing 30. The opening 31c is provided in a side face of the housing 30 (in this example the lower surface). The opening 31a and the opening 31b overlap each other.

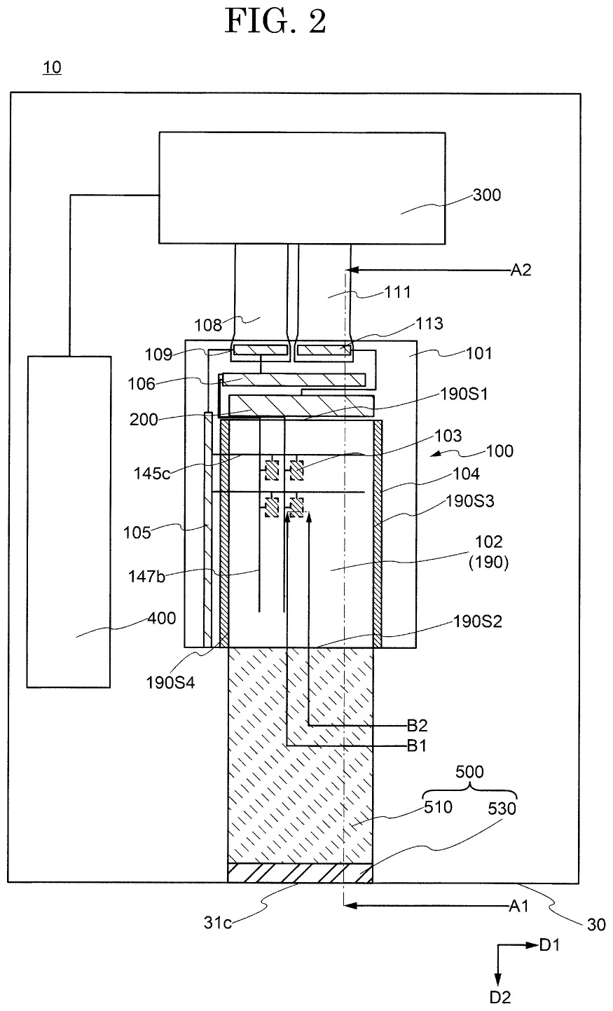

FIG. 2 is a schematic diagram showing an inside of the housing of the display system 10. As shown in FIG. 2, the display system 10 includes a display panel 100, a lighting device 200, a control device 300, a power supply device 400, and an optical component 500. The housing 30 surrounds the display panel 100, the lighting device 200, the control device 300, the power supply device 400, and the optical component 500.

The display panel 100 includes a substrate 101, a display region 102, a pixel 103, a light shielding member 104, a driving circuit 105, a driving circuit 106, a flexible printed circuit board 108, a terminal part 109, and a substrate 190.

The driving circuit 105 has a function as a gate driver. The driving circuit 106 has a function as a source driver. In the display region 102, a plurality of pixels 103 are arranged apart from each other in a grid pattern. The pixel 103 functions as a component of an image. Specifically, the pixel 103 includes a liquid crystal element 130 to be described later. The liquid crystal element 130 has a function of transmitting or scattering light. A scan line 145c is connected to the driving circuit 105. A signal line 147b is connected to the driving circuit 106. The pixel 103 is connected to the scan line 145c and the signal line 147b.

The lighting device 200 includes a light emitting element 220. The light emitting element 220 includes a plurality of light emitting elements 220. In this example, a light emitting diode is used as the light emitting element. The light emitted from the light emitting element 220 is applied to the display panel 100 (specifically, the substrate 190).

The control device 300 includes an arithmetic element and a memory element. A CPU (Central Processing Unit), an ASIC (Application Specific Integrated Circuit), or an FPGA (Field-Programmable Gate Array) is used as the arithmetic element. A DRAM (Dynamic Random Access Memory), a SSD (Solid State Drive), or a hard disk is used as the memory element. The control device 300 is connected to the display panel 100 at the terminal part 109 via the flexible printed circuit board 108. The control device 300 is connected to the lighting device 200 at a terminal part 113 via a flexible printed circuit board 111.

The power supply device 400 supplies power to the display panel 100, the lighting device 200, and the control device 300. In this example, although a lithium-ion battery is used for the power supply device 400, an optimal battery may be used as appropriate.

The optical component 500 is provided corresponding to a second side face 190S2 of the substrate 190 in the display panel 100 (the display region 102). Specifically, the optical component 500 is provided between the second side face 190S2 of the substrate 190 and the opening 31c. The optical component 500 includes a light guide member 510 and a diffusion member 530. The diffusion member 530 is provided in the opening 31c.

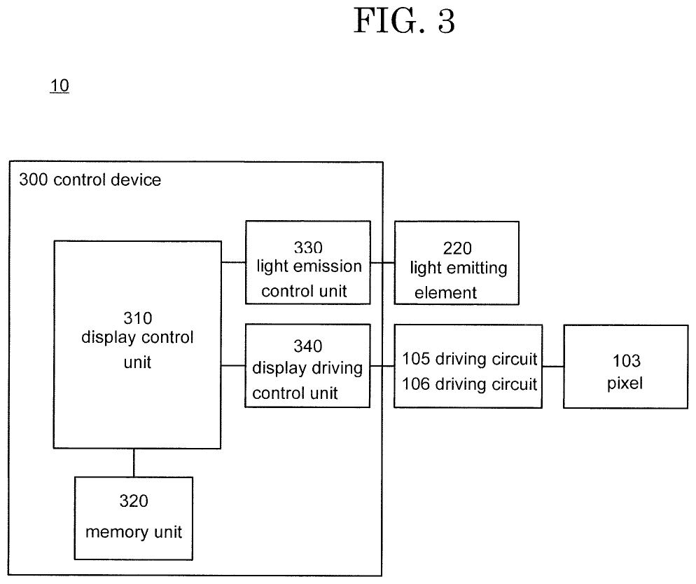

FIG. 3 is a functional block diagram of the display system 10. The control device 300 includes a display control unit 310, a memory unit 320, a light emission control unit 330, and a display driving control unit 340.

The display control unit 310 is composed of various logic circuits. The display control unit 310 controls display information stored in the memory unit 320 (or information stored in an external device) based on programs stored in the memory unit 320. The display control unit 310 may initiate the display control process in response to receiving the input information.

The light emission control unit 330 functions as a switch for driving the light emitting element 220 based on an instruction from the display control unit 310. The driving method of the light emitting element 220 will be described later.

The display driving control unit 340 transmits a display control signal to the driving circuit 105 and the driving circuit 106 based on the instruction from the display control unit 310.

In FIG. 2 and FIG. 3, the display control unit 310 drives the light emitting element 220 via the light emission control unit 330. The display control unit 310 inputs the display control signal to the driving circuit 105 and the driving circuit 106 via the display driving control unit 340 and the flexible printed circuit board 108. Next, a scan signal from the driving circuit 105 is transmitted to the pixel 103 in the display region 102 via the scan line 145c. Similarly, a video signal from the driving circuit 106 is transmitted to the pixel 103 in the display region 102 via the signal line 147b. As a result, a still image and moving image are displayed on the display region 102 of the display panel 100.

The light emitting element 220 includes a first color (e.g., red) light emitting element 220R, a second color (e.g., green) light emitting element 220G, and a third color (e.g., blue) light emitting element 220B. The light emission control unit 330 controls each of the first color light emitting element 220R, the second color light emitting element 220G, and the third color light emitting element 220B to emit light in time division based on the light source control signal. Thus, the first color light emitting element 220R, the second color light emitting element 220G, and the third color light emitting element 220B are driven in a field sequential method.

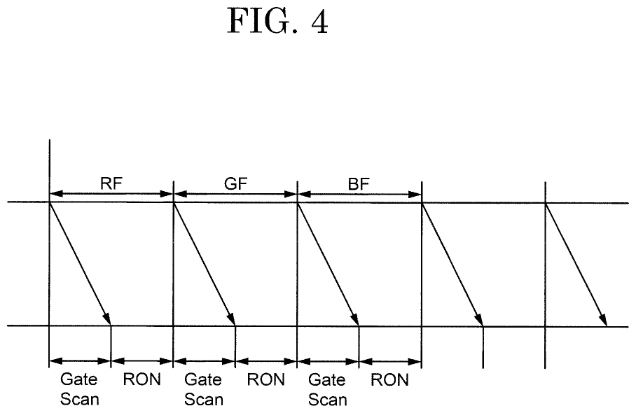

FIG. 4 is a timing chart for explaining the timing at which the light emitting element 220 emits light. As shown in FIG. 4, in the first sub-frame (first predetermined time) RF, the first color light emitting element 220R emits light in a first color light emitting period RON the light is scattered in the pixel 103 which is selected within one vertical scanning period GateScan by which a still image or moving image is displayed. In the entire display panel 100, if the gradation signal corresponding to the output gradation value of each pixel 103 is supplied to each signal line 147b described above in the pixel 103 selected in one vertical scanning period GateScan, only the light of the first color light is on in the first color light emitting period RON.

Next, in the second sub-frame (second predetermined time) GF, the second color light emitting element 220G emits light in the second color light emitting period GON, the light is scattered in the pixel 103 which is selected within one vertical scanning period GateScan, and a still image or moving image is displayed. In the entire display panel 100, if the gradation signal corresponding to the output gradation value according to each pixel 103 is supplied to each signal line 147b described above in the pixel 103 selected in one vertical scanning period GateScan, only the light of the second color light is on in the second color light emitting period GON.

Furthermore, in the third sub-frame (third predetermined time) BF, the third color light emitting element 220B emits light in the third color light emitting period BON, the light is scattered in the pixel 103 which is selected within the one vertical scanning period GateScan, and a still image or moving image is displayed. In the entire display panel 100, if the gradation signal corresponding to the output gradation value of each pixel 103 is supplied to each signal line 147b described above in the pixel 103 selected in one vertical scanning period GateScan, only the light of the third color light is on in the third color light emitting period BON.

Since human eyes have a temporal resolution limitation and an afterimage is generated, a composite image of three colors is recognized in a period of one frame. In the field sequential method, the color filter can be eliminated. Thus, the absorption loss of light in the color filter is reduced. Therefore, a higher transmittance can be realized in the display panel. In a color filter method, one pixel is formed by sub-pixels obtained by dividing the pixel 103 for each of the first color, the second color, and the third color, whereas in the field sequential method, such sub-pixel division is not necessary. The fourth sub-frame may further be provided to emit light of the fourth color different from the light of the first color, the light of the second color, and the light of the third color.

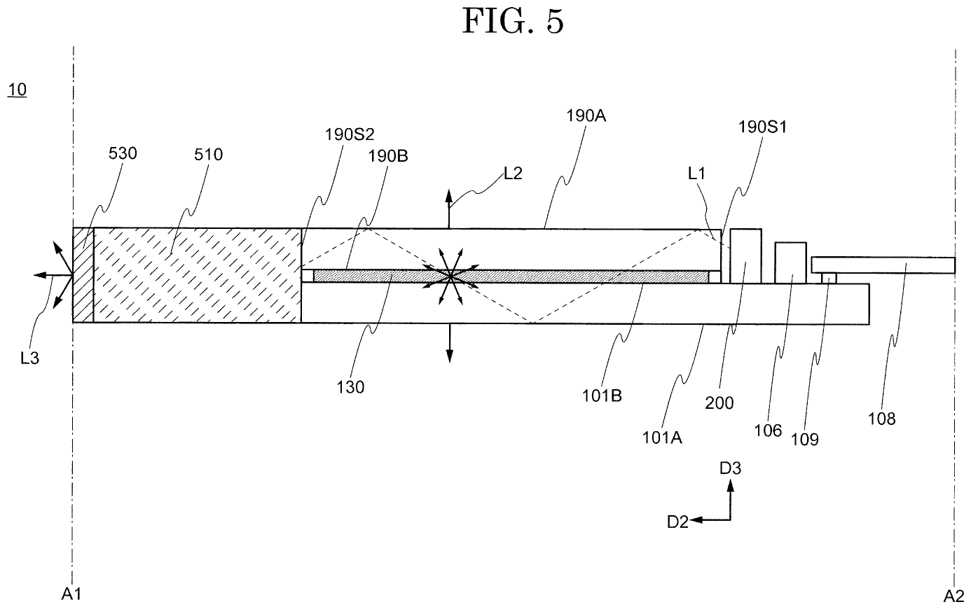

FIG. 5 is a cross-section A1-A2 of FIG. 2. As shown in FIG. 5, the lighting device 200 faces the first side face 190S1 of the substrate 190 (also referred to as a counter substrate). As shown in FIG. 5, the lighting device 200 irradiates the first side face 190S1 of the substrate 190 with the light L1. The irradiated light L1 is incident on the display panel 100 from the first side face 19051. The first side face 19051 is a light-incident surface.

A material having light transmittance is used as the substrate 101 and the substrate 190. For example, a glass substrate is used as the substrate 101 and substrate 190. A quartz substrate or organic resin substrate may be used as the substrate 101.

As shown in FIG. 5, the light L1 irradiated from the lighting device 200 propagates in the direction (second direction D2) away from the first side face 19051 while being reflected at a first main surface 101A of the substrate 101 (also referred to as an array substrate) and a first main surface 190A of the substrate 190. When the light L1 is directed to the outside from the first main surface 101A of the substrate 101 or the first main surface 190A of the substrate 190, the light L1 proceeds from a medium having a large refractive index to a medium having a small refractive index. Therefore, if the incident angle of the light L1 incident on the first main surface 101A of the first substrate 101 or the first main surface 190A of the substrate 190 is greater than a critical angle, the light L1 is completely reflected at the first main surface 101A of the substrate 101 or the first main surface 190A of the substrate 190.

As shown in FIG. 5, the light L1 propagated inside the substrate 101 and the substrate 190 is scattered by the pixel 103 having scattered liquid crystal molecules, and the incident angle of the scattered light is an angle smaller than the critical angle. As a result, the light L2 is emitted to the outside from the first main surface 190A of the substrate 190 and the first main surface 101A of the substrate 101, respectively. The light L2 emitted from the first main surface 190A of the substrate 190 and the first main surface 101A of the substrate 101 to the outside is observed by an observer.

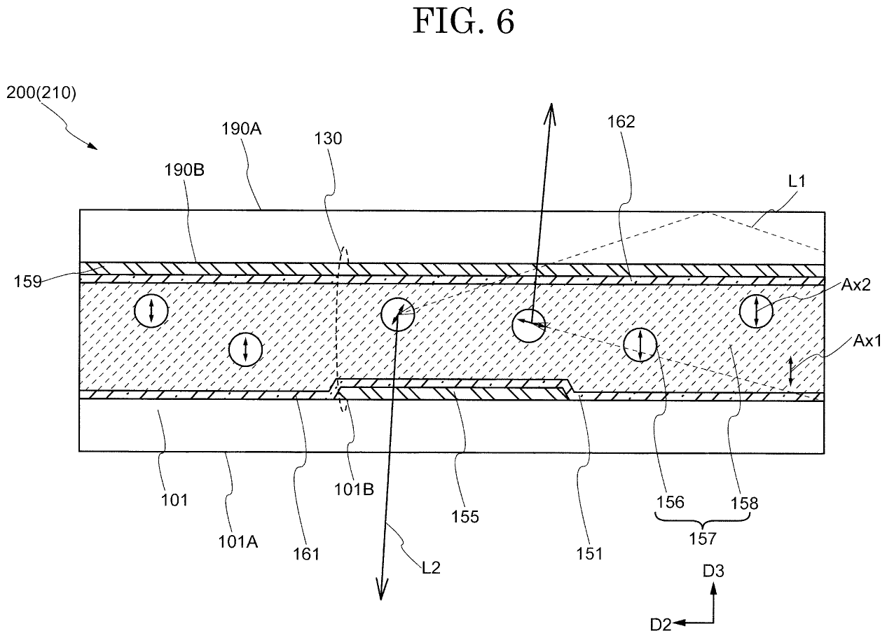

FIG. 6 is an enlarged cross-sectional view of a part of the display panel 100. In FIG. 6, a pixel electrode 155, a liquid crystal layer 157, and a common electrode 159 are used for the liquid crystal element 130.

The pixel electrode 155 has a function as the first electrode of the liquid crystal element 130. A conductive material having light transmittance is used for the pixel electrode 155. For example, an oxide conductive material such as ITO and IZO is used for the pixel electrode 155.

The common electrode 159 functions as the second electrode of the liquid crystal element 130. The common electrode 159 is provided to continuously cover the pixel electrode 155 across a plurality of pixel electrodes 155. A material having light transmittance and conductivity is provided on the common electrode 159. For example, an oxide conductive material such as ITO and IZO is used for the common electrode 159.

The liquid crystal layer 157 includes a polymer 158 and liquid crystal molecules 156. Since the liquid crystal molecules 156 are dispersed in the polymer 158, the liquid crystal layer is a polymer-dispersed liquid crystal. The polymer 158 and the liquid crystal molecules 156 have optical anisotropy, respectively. A sealing material is provided at the end portion of the liquid crystal layer 157. A transparent resin material is used for the sealing material.

A first alignment film 161 is provided on the substrate 101. A second alignment film 162 is provided on the substrate 190. The first alignment film 161 and the second alignment film 162 are, for example, vertical alignment films.

The orientation of the liquid crystal contained in the liquid crystal molecules 156 is controlled by the voltage difference between the pixel electrode 155 and the common electrode 159. The orientation of the liquid crystal changes with the voltage applied to the pixel electrode 155. As the orientation of the liquid crystal changes, the degree of scattering of light passing through the pixel 103 changes.

As shown in FIG. 6, in the state where no voltages are applied between the pixel electrode 155 and the common electrode 159, the directions of the optical axis Ax1 of the polymer 158 and the optical axis Ax2 of the liquid crystal molecules 156 are equal to each other. The optical axis Ax2 of the liquid crystal molecules 156 is parallel to third direction D3 of the liquid crystal layer 157. The optical axis Ax1 of the polymer 158 is parallel to the third direction D3 of the liquid crystal element 130 regardless of the presence or absence of the voltage.

The refractive indexes of the polymer 158 and the liquid crystal molecules 156 are equal to each other. In the state where no voltage is applied between the pixel electrode 155 and the common electrode 159, the refractive index differences between the polymer 158 and the liquid crystal molecules 156 are zero in all directions. In this case, the liquid crystal layer 157 is in a non-scattering state that does not scatter the light L1. The light L1 propagates in a direction (second direction D2) away from the lighting device 200 (the light emitting element 220) while being reflected at the first main surface 101A of the substrate 101 and the first main surface 190A of the substrate 190. When the liquid crystal layer 157 is in the non-scattering state that does not scatter the light L1, the background of the first main surface 190A side of the substrate 190 is visually recognized from the first main surface 101A of the substrate 101, and the background of the first main surface 101A side of the substrate 101 is visually recognized from the first main surface 190A of the substrate 190.

On the other hand, between the pixel electrode 155 and the common electrode 159 where the voltage is applied, the optical axis Ax2 of the liquid crystal molecules 156 will be inclined by an electric field generated between the pixel electrode 155 and the common electrode 159. Since the optical axis Ax1 of the polymer 158 is not changed by the electric field, the direction of the optical axis Ax1 in the polymer 158 and the direction of the optical axis Ax2 in the liquid crystal molecules 156 are different from each other. In this case, the light L1 is scattered in the pixel 103 where the pixel electrode 155 is arranged to which the voltage is applied. As described above, the light L2, which is a part of the scattered light L1 emitted from the opening 31a and the opening 31b to the outside through the first main surface 101A of the substrate 101 or the first main surface 190A of the substrate 190, is observed by the observer. As a result, the image displayed on the display panel 100 is visible from the front face and the back face of the housing.

Referring back to FIG. 2, the light shielding member 104 is provided on the third side face 190S3 and the fourth side face 190S4 which are sides in the longitudinal direction of the substrate 190 (second direction D2). The third side face 190S3 and the fourth side face 190S4 are sides in the longitudinal direction of the substrate 190 (second direction D2). Therefore, light is not emitted from the third side face 190S3 and the fourth side face 190S4.

On the other hand, a light shielding member is not provided on the second side face 190S2 facing the first side face 190S1. The second side face 190S2 is a side in the short direction (first direction D1) of the substrate 190. Instead of the light shielding member, the light guide member 510 is provided facing the second side face 190S2. In this case, as shown in FIG. 5, the light L1 reaching the second side face 190S2 of the substrate 190 is emitted to the light guide member 510. The light L1 incident on the light guide member 510 is guided to the diffusion member 530. The light L1 guided to the diffusion member 530 is emitted as the light L3 to the outside from the opening 31c. It is desirable that the amount of light emitted from the opening 31c is, for example, between 40% and 60% of the amount of light emitted from the lighting device 200. As a result, in the case where an article is provided under the opening 31c, the article can be illuminated with optimum illumination intensity while maintaining brightness required for displaying an image.

Therefore, by using the present embodiment, the displayed image can be visually recognized from the front face and the back face of the housing, and light can be extracted from the bottom of the housing. That is, it is possible to improve the light utilization efficiency by using the present embodiment.

### (1-2. Cross-Sectional Configuration of Display Panel)

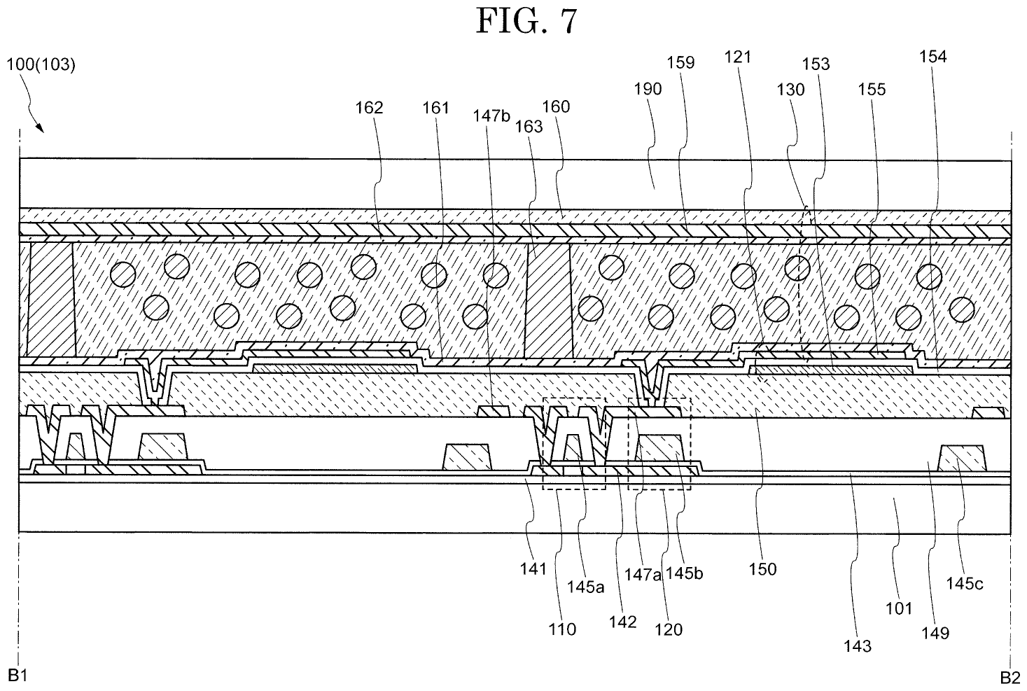

Next, the cross-sectional configuration of the display panel 100 will be described in detail. As shown in FIG. 7, the pixel 103 of the display panel 100 includes a transistor 110, a capacitive element 120, a capacitive element 121, and the liquid crystal element 130. The display panel 100 includes an insulating layer 141, an insulating layer 149, a planarization layer 150, and an adhesive layer 160 in addition to the above.

The transistor 110 has a semiconductor layer 142, a gate insulating layer 143, a gate electrode 145a, and a source-drain electrode 147a. Although the transistor 110 has a top gate/top contact structure, the present invention is not limited thereto. For example, the transistor 110 may be a bottom gate structure or a bottom contact structure.

A source or drain region of the semiconductor layer 142 and a capacitance electrode 145b are used for the capacitive element 120 with the gate insulating layer 143 as a dielectric. A conductive layer 153 and the pixel electrode 155 are used for the capacitive element 121 with an insulating layer 154 as a dielectric.

The insulating layer 141 is provided on the substrate 101. The insulating layer 141 has a function as a base film. This can suppress the diffusion of impurities, typically alkaline metal, water, hydrogen, and the like, from the substrate 101 to the semiconductor layer 142.

The semiconductor layer 142 is provided on the insulating layer 141. The semiconductor layer 142 may be a silicone, an oxide semiconductor, or an organic semiconductor, or the like.

The gate insulating layer 143 is provided on the insulating layer 141 and the semiconductor layer 142. The gate insulating layer 143 may be silicon oxide, silicon oxynitride, silicon nitride, or other high dielectric constant inorganic material.

The gate electrode 145a is provided on the gate insulating layer 143. The gate electrode 145a is connected to the scan line 145c as appropriate. The gate electrode 145a and the capacitance electrode 145b are also provided on the gate insulating layer 143. The gate electrode 145a, the capacitance electrode 145b, and the scan line 145c are formed of conductive materials selected from tantalum, tungsten, titanium, molybdenum, aluminum, and the like. The gate electrode 145a and the capacitance electrode 145b may be a single-layer structure of the above-described conductive materials or may be a stacked structure.

The source-drain electrode 147a is provided on the insulating layer 149. The source-drain electrode 147a is connected to the signal line 147b as appropriate. Materials similar to those listed as examples of materials of the gate electrode 145a are used for the source-drain electrode 147a and the signal line 147b. In this case, the same material as that of the gate electrode 145a may be used for the source-drain electrode 147a, or different materials may be used for the source-drain electrode 147a.

The same material as that of the gate insulating layer 143 is used for the insulating layer 149. The insulating layer 149 is provided on the gate insulating layer 143, the gate electrode 145a, and the capacitance electrode 145b. The insulating layer 149 may be a single layer or a stacked structure of the above materials.

The planarization layer 150 is provided on the insulating layer 149. The planarization layer 150 functions as a planarization film. The planarization layer 150 contains an organic resin. In this example, an acrylic resin is used for the planarization layer 150. The planarization layer 150 is not limited to an acrylic resin, and an epoxy resin, a polyimide resin, a polyamide resin, a polystyrene resin, a polyethylene resin, a polyethylene terephthalate resin, or the like may be used for the planarization layer 150. Lamination of an organic resin and an inorganic material may be used for the planarization layer 150.

The conductive layer 153 is provided on the planarization layer 150. A material having light transmittance is used for the conductive layer 153. For example, an oxide conductive material such as ITO or an IZO is used for the conductive layer 153. In addition to the conductive layer 153, other wirings (not specifically shown) bonded to the source-drain electrode 147a are also formed using the same conductive material.

The insulating layer 154 is provided on the planarization layer 150 and the conductive layer 153. The same material as that of the gate insulating layer 143 is used for the insulating layer 154.

The liquid crystal layer 157 is provided with a spacer 163. The spacer 163 maintains the distance between the pixel electrode 155 and the common electrode 159.

An inorganic material, an organic material, or a composite material of an organic material and an inorganic material is used for the adhesive layer 160. For example, an acrylic resin is used for the adhesive layer 160.

### Second Embodiment

In the present embodiment, a display system different from the first embodiment will be described. Specifically, a display system having a wavelength converter will be described.

### (2-1. Configuration of Display System)

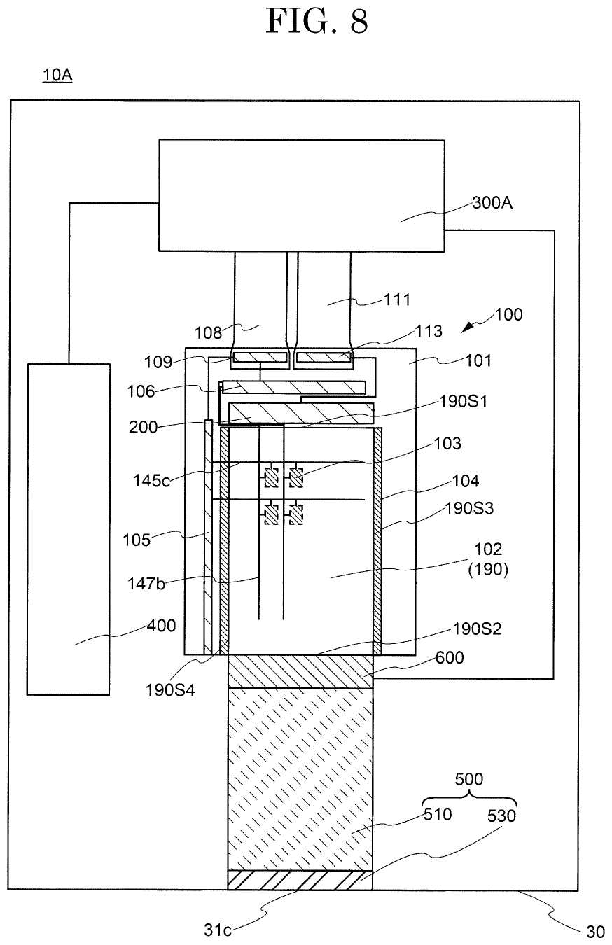

FIG. 8 is a schematic diagram showing the inside of a housing in a display system 10A. As shown in FIG. 8, the display system 10A includes a wavelength converter 600 in addition to the display panel 100, the lighting device 200, a control device 300A, the power supply device 400, and the optical component 500. The wavelength converter 600 is provided corresponding to the second side face 190S2 of the substrate 190. More specifically, the wavelength converter 600 is provided between the second side face 190S2 of the substrate 190 and the optical component 500. The light L1 emitted from the display panel 100 is incident on the wavelength converter 600.

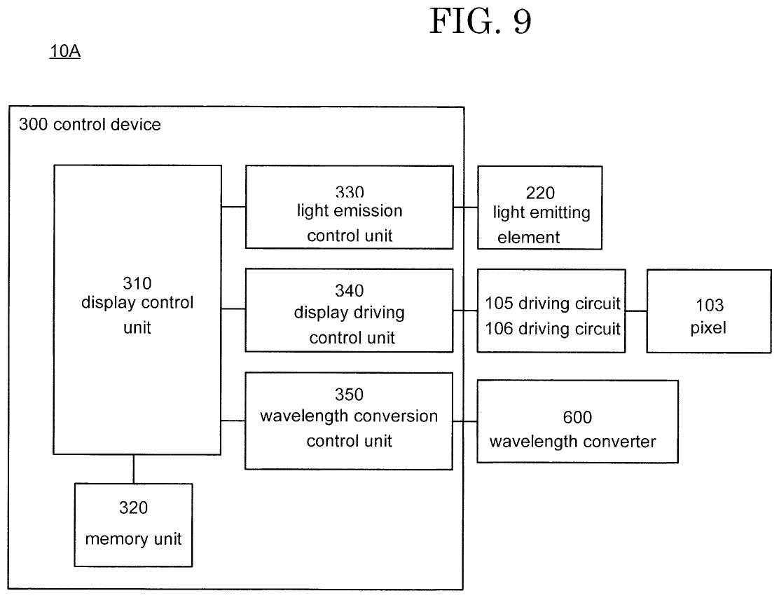

FIG. 9 is a functional block diagram of the display system 10A. The control device 300A includes a wavelength conversion control unit 350 in addition to the display control unit 310, the memory unit 320, the light emission control unit 330, and the display driving control unit 340.

The wavelength converter 600 converts the wavelength of the incident light L1 according to the control by the wavelength conversion control unit 350. In this example, the wavelength band of the converted light L1 may be an infrared wavelength band or an ultraviolet wavelength band. A fourth sub-frame may be further provided in the case where light in the ultraviolet wavelength band or the infrared band is emitted. In this case, a light emitting element that emits ultraviolet light or infrared light as the light of the fourth color different from the light of the first color, the light of the second color, and the light of the third color may be provided.

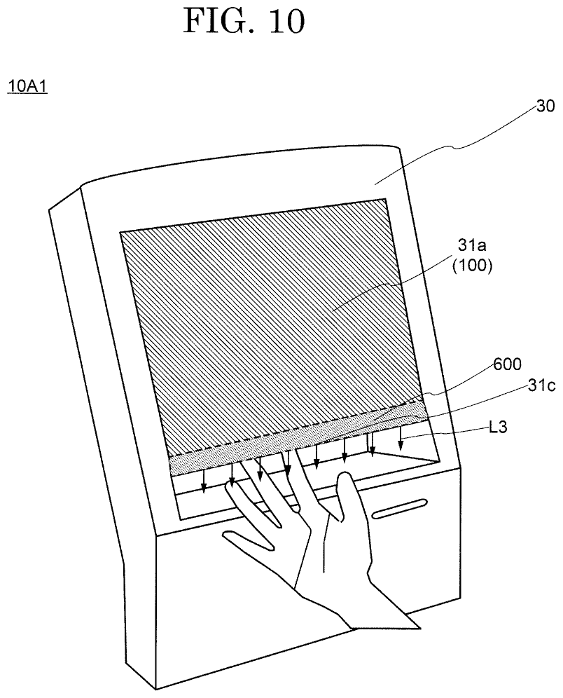

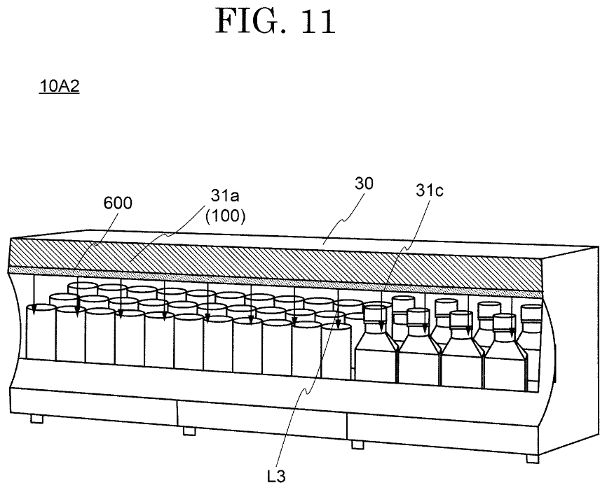

FIG. 10 and FIG. 11 are specific examples of the display system of the present embodiment.

In FIG. 10, the housing 30 of a display system 10A1 has the shape of a monitor. An image displayed by the display panel 100 can be visually recognized from the opening 31a. The light L3 is emitted from the opening 31c. The light L3 is converted into ultraviolet rays by the wavelength converter 600. In this case, the wavelength band of the light L3 is preferably 200 nm or more and 300 nm or less. More specifically, the peak wavelength of the light L3 is preferably 253.7 nm. This is because the sterilization effect by the light L3 is enhanced. In the display system 10A1, when a hand is held on the lower side of the opening 31c of the housing 30, the hand can be sterilized. In other words, by using the present embodiment, sterilization and disinfection of the hand can be performed while visually recognizing the display screen.

In FIG. 11, the housing 30 of a display system 10A2 has the shape of a beverage storage fixture. The opening 31a is provided on the front upper portion of the housing 30. A user can visually recognize the displayed image displayed on the display panel 100 in the opening 31a. The light L3 is emitted from the opening 31c. The light L3 is converted into infrared by the wavelength converter 600. In this case, the wavelength band of the light L3 is preferably 0.78 μm or more and 1 mm or less. In the display system 10A2, an object (a drink in this case) provided on the lower side of the opening 31c of the housing 30 is warmed by the light L3. That is, by using the present embodiment, it is possible to warm the product while visually recognizing the display screen.

### Third Embodiment

In the present embodiment, a display system different from the first embodiment will be described. Specifically, a display system having a wavelength converter and a sensor will be described.

### (3-1. Configuration of Display System)

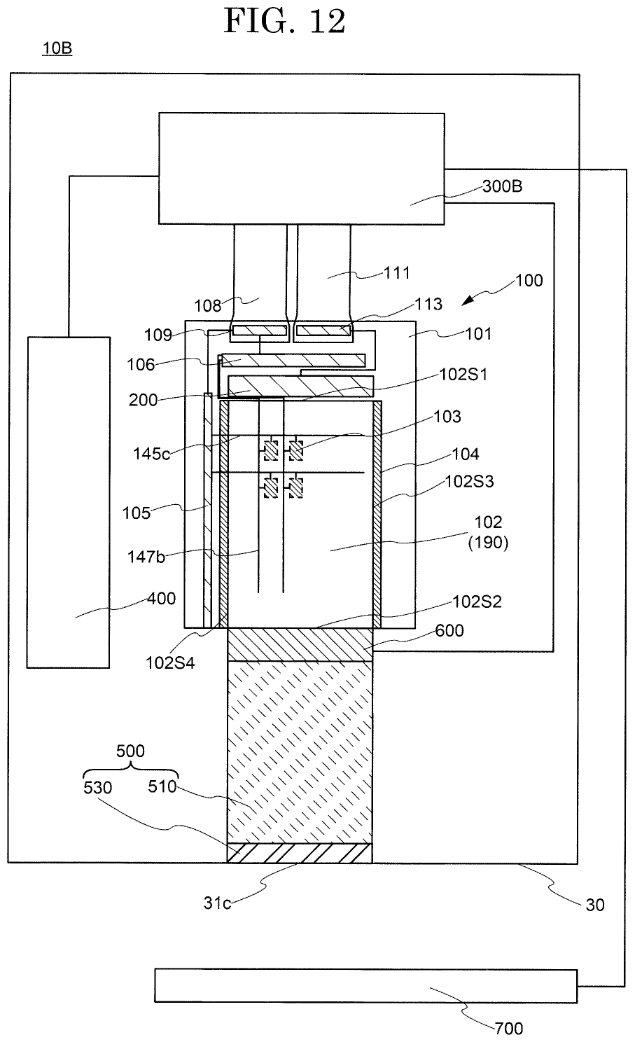

FIG. 12 is a schematic diagram showing an inside of a housing of a display system 10B. As shown in FIG. 12, the display system 10B includes the wavelength converter 600 and a sensor 700 in addition to the display panel 100, the lighting device 200, a control device 300B, the power supply device 400, and the optical component 500.

The wavelength converter 600 is provided between the display panel 100 and the optical component 500. The light L1 emitted from the display panel 100 is incident on the wavelength converter 600.

The sensor 700 is provided on the outside of the housing 30. The sensor 700 faces the opening 31c. The sensor 700 may be a CCD (Charge Coupled Device) image sensor or a CMOS (Complementary Metal Oxide Semiconductor) image sensor.

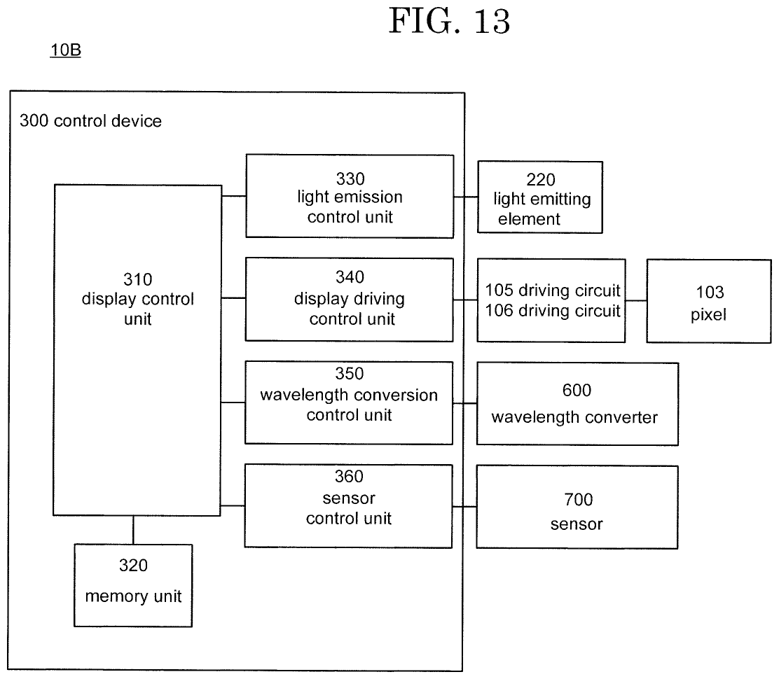

FIG. 13 is a functional block diagram of the display system 10B. The control device 300B includes the wavelength conversion control unit 350 and a sensor control unit 360 in addition to the display control unit 310, the memory unit 320, the light emission control unit 330, and the display driving control unit 340.

The wavelength converter 600 converts the wavelength of the incident light L1 according to the control by the wavelength conversion control unit 350. In this example, the wavelength band of the converted light L1 may be an infrared wavelength band.

The sensor 700 detects the light L3 emitted from the opening 31c. In this example, the sensor 700 detects infrared light. The sensor control unit 360 may generate various information according to the amount of the light L3 received by the sensor 700.

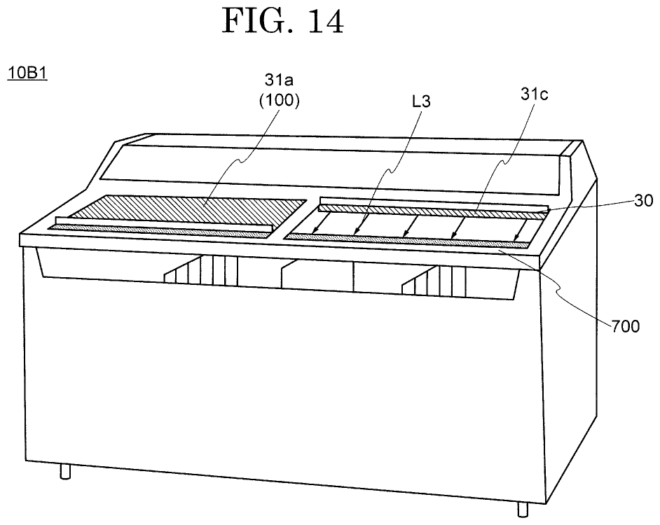

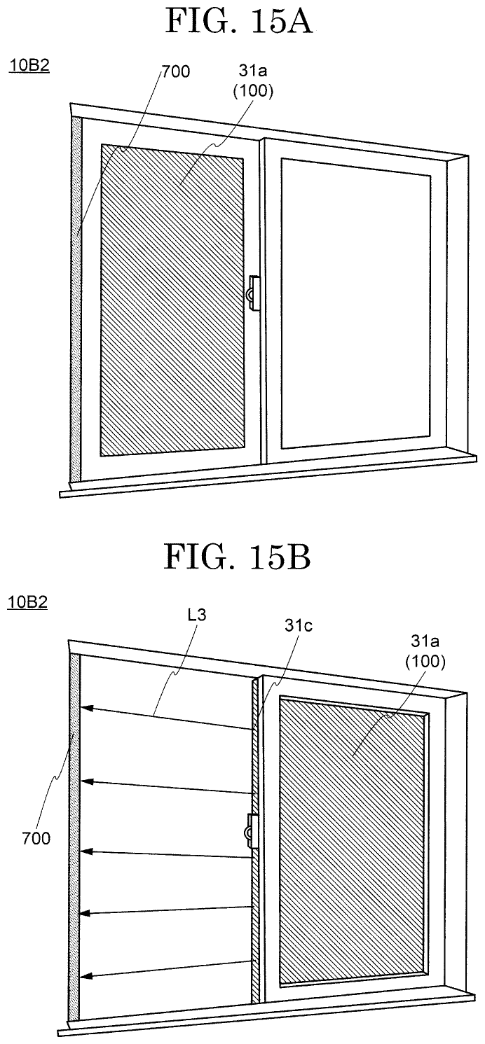

FIG. 14, FIG. 15A and FIG. 15B are specific examples of the display system of the present embodiment.

In FIG. 14, the housing 30 of a display system 10131 has the shape of a freezer showcase. A portion corresponding to the opening 31a of the housing 30 is provided in a translucent glass portion of a slide opening and closing door. The user can visually recognize the displayed image displayed on the display panel 100 in the opening 31a. In the display system 10B, the light L3 is emitted when the opening 31c leaves the end portion of the showcase. In this case, the light L3 is converted into infrared by the wavelength converter 600. In the present embodiment, the light emitted from the opening 31c is detected by the sensor 700. As a result, it is possible to detect the opening and closing state of the slide door of the freezer showcase.

In FIG. 15A and FIG. 15B, the housing 30 of a display system 1062 has the shape of a residential window. The portion corresponding to the opening 31a of the housing 30 is provided in the window portion. As shown in FIG. 15A, the user can visually recognize the displayed image displayed on the display panel 100 in the opening 31a. As shown in FIG. 15B, the light L3 is emitted when the opening 31c leaves the side end portion of the window in the display system 10B. In this case, the light L3 is converted into infrared by the wavelength converter 600. In the present embodiment, the light emitted from the opening 31c is detected by the sensor 700. As a result, it is possible to detect the opening and closing state of the window.

In the present embodiment, when a finger is interposed between the opening 31c and the sensor 700, vein authentication can be performed using the light transmitted through the finger.

That is, the display system can have a sensing function while visually recognizing the display screen by using the present embodiment.

In the present embodiment, although an example is shown in which the sensor is provided on the outside of the housing 30 and faces the opening 31c, the present invention is not limited to this. For example, the sensor may be provided inside the housing 30. In this case, the sensor 700 may detect the reflected light with respect to the light incident on the object. As a result, it is possible to detect the distance between the sensor 700 and the object, fingerprint or pulse, and the like.

### (Modifications)

Within the spirit of the present invention, it is understood that various modifications and changes can be made by those skilled in the art and that these modifications and changes also fall within the scope of the present invention. For example, the addition, deletion, or design change of components, or the addition, deletion, or condition change of processes as appropriate by those skilled in the art based on each embodiment are also included in the scope of the present invention as long as they are provided with the gist of the present invention.

In the first embodiment of the present invention, although an example is shown in which the optical component 500 (the light guide member 510 and the diffusion member 530) is provided, the present invention is not limited thereto. For example, one of the light guide member 510 and the diffusion member may be provided depending on the position of the display panel 100, or other optical components may be provided, and the optical component 500 does not necessarily have to be provided.

What is claimed is:

1. A display system comprising:
a display panel which displays images;
a lighting device facing a first side face of the display panel; and
a housing surrounding the display panel and the lighting device, the housing including a front face facing the display panel, a side face, a first opening provided in the front face, and a second opening in the side face, wherein
a light emitted from the lighting device enters the display panel from the first side face,
a first portion of the light is emitted from the first opening, and a second portion of the light is emitted from the second opening through at least one portion of a second side face of the display panel, the second side face facing the first side face.

2. The display system according to claim 1, wherein
the housing includes a third opening provided in the back face facing the display panel in the housing, and
an image displayed on the display panel is visible from the front face and the back face.

3. The display system according to claim 1, further comprising:
an optical member provided facing the second side.

4. The display system according to claim 1, further comprising:
a wavelength converter facing to the second side face and converting a wavelength of light emitted from the second side face.

5. The display system according to claim 4, wherein
a wavelength range of light converted by the wavelength converter is in an infrared wavelength range or ultraviolet wavelength range.

6. The display system according to claim 1, further comprising:
a sensor facing the second opening and receiving light emitted from the second opening.

7. The display system according to claim 1, wherein
an amount of light emitted from the second opening is between 40% and 60% of an amount of light emitted from the lighting device.
